(19) United States
(12) Patent Application Publication
(10) Pub. No.: US 2021/0076941 A1
(43) Pub. Date: Mar. 18, 2021

(54) Apparatus for Real Time Evaluation of Tissue During Surgical Ablation Procedures

(71) Applicant: Kurt Daniel Van Laar, Simi Valley, CA (US)

(72) Inventor: Kurt Daniel Van Laar, Simi Valley, CA (US)

(21) Appl. No.: 16/572,469

(22) Filed: Sep. 16, 2019

(51) Int. Cl. A61B 5/00; G01N 1/04; G01N 21/05; G01N 21/3504; G01N 21/17; G06N 3/04; G01N 33/497

(52) U.S. Cl. CPC A61B 5/0082; G01N 1/04; G01N 21/05; G01N 2021/3595; G01N 21/1702; G06N 3/0436; G01N 33/497; G01N 21/3504

(57) ABSTRACT

An apparatus for the real time evaluation of in vivo tissue ablation is provided. The apparatus comprises a Near Infrared illumination source that delivers light to one side of a windowed flow-thru cuvette and exit to a Fourier-Transform Infrared [FTIR] spectrometer opposite the light source. The light traversal is via fiberoptic cables. While surgical smoke traverses the cuvette, the smoke is subjected to continuous FTIR sampling and those samples are compared in real time to a database of known cancer, necrotic or diseased tissue spectrums, utilizing Artificial Intelligence [AI] software. The apparatus in real-time indicates to the surgeon weather the ablation smoke contains cancerous or normal tissue via LED's, sounds, or tactile indicators.

### CROSS-REFERENCE TO RELATED APPLICATIONS

This application claims priority to U.S. provisional patent application No. 62/921,204, filed on Jun. 5, 2019 by the applicant.

### DESCRIPTION

### Field of the Invention

The present invention relates to devices, systems, and methods for analyzing, localizing, and/or identifying tissues. More specifically, the present invention relates to devices, systems, and methods for analyzing, localizing, or identifying tissues in real-time and in vivo by evaluating the smoke generated by tissues ablation and doing real-time data analysis of the resulting smoke using Fast Fourier Transform InfraRed [FTIR] spectrometry and spectral evaluation software.

### Referenced Art

PRINGLE et al. US 2018/0042583 A1; Pringle mentions FTIR spectroscopy in claim 199, 211 primally as an aside with no mention of comparison of normal vs abnormal real-time spectral analysis or the actual utilization of FTIR data.

LINFORTH, et al. U.S. Pat. No. 5,869,344; Linforth discusses the use of mass-spectrometers in doing gas analysis, there is no mention of smoke of FTIR technology.

WISEMAN et al. U.S. Pat. No. 8,324,570 B2; Wisemans patent primally relates to a sampling probe with the mention that the extracted analyte could be fed to an IR or florescent spectrophotometer without the mentioning of smoke or FTIR throughout the patent.

DAVIS et al. U.S. Pat. No. 10,219,736 B2; Davis discusses utilizing smart phones to provide images that are processed using a data base and using between 4 and 20 color input channels to provide image matching.

TAKATS U.S. Pat. No. 9,046,448 B2; Takats discusses surgical smoke within the body of the discussion of FIG. 1 and example 1 with the goal of providing a sample to the ion trap of a mass spectrometer [600-900 nm] not within the range of an FTIR spectrometer [1,300-2,600 nm]

TAKATS U.S. Pat. No. 10,335,123 B2; Takats discusses ionized surgical smoke within the abstract with the goal of providing a sample to the ion trap of a mass spectrometer [600-900 nm] not within the range of an FTIR spectrometer [1,300-2,600 nm] and not in real-time.

### BACKGROUND OF THE INVENTION

While Mohs “Micrographic surgery” is the Gold Standard in dermatological surgery, The dermatological community needs a less time-consuming method of removal of skin cancers than Mohs. Currently these diathermy procedures have been augmented with carbon dioxide and Erbium YAG lasers. However, by using lasers, the process may prove more difficult especially visually discerning cancer cells from non-cancerous tissue.

With the ever-growing demands on the medical profession, an interactive real time method of laser and diathermy ablation tissue type detection of cancerous verses non-cancerous tissue would save many hours over the Mohs or other microscopic methods.

Typically, surgical smoke is considered hazardous requiring vacuum pumps and filters to mitigate [FDA 21CFR878.5050]. This patent puts the smoke to good use while still mitigating the hazard using single use flow-thru disposable cuvettes with an integral filter and single use tubing.

This invention creates a surgical assisting tool applicable for use in dermatologic, oncologic, laparoscopic, and other surgeries as defined in [FDA 21CFR878.4400], where healthy tissue is adjacent to cancerous, necrotic, or diseased tissue. This apparatus provides real time sampling of the smoke created by laser ablation or electrosurgery, providing the ability to differentiate between cancer and non-cancerous tissue products within the smoke, in real time by using an Infrared Fast Fourier Transform Spectrometer [FTIR] to sample and classify the surgical smoke.

### SUMMARY OF THE INVENTION

The present invention provides a system for analyzing, localizing, and identifying tissue types [cancer vs. non-cancerous] in real time i.e. sub seconds as opposed to multiple minutes using mass spectrometry or microscope and stains. This devices, systems and methods of the present invention are capable of providing in situ and in vivo investigations of biological tissues while doing no harm to the individual receiving this procedure, and provides real-time feedback during procedures, such as surgical procedures.

### Advantages

Advantages of the invention during surgery include:
1. Providing rea-time indication of the type of tissue being ablated, cancerous vs non-cancerous which keeps removal of healthy tissue to a minimum.
2. Allowing surgeons to detect how different types of tissues are distributed over surgical area.

This feature increases detection of tumor spots, increases the precision of tissue removal in case of tumor removal surgeries and removal of necrotized/ischemic tissues, and minimizes the mass of healthy tissues removed.
3. Enable the real-time, in-vivo & in-situ identification of tissue health during surgery.
4. Provides data logging during procedures to create a real time record and update the tissue data base.

### BRIEF DESCRIPTION OF THE DRAWINGS

The features and advantages of the invention will be apparent to those of ordinary skill in the art from the following detailed description of which:

FIG. 1 is a schematic drawing showing the components of the present invention in its initial configuration. Where: The computer 10, disposable vacuum hose 20, Disposable flow thru Cuvette 25, vacuum pump 30, Vacuum pump discharge 40, Surgical waste treatment container 50, FTIR spectrometer 60, Fiberoptic cable to spectrometer 65, Cuvette holder 70, Fiberoptic cable from light source 75, Calibrated light source 80, Disposable vacuum tube to cuvette 90, Smoke pickup with indicators, 100, Control cabling 110.

FIG. 2 depicts the cuvette holder 70, with a cuvette 25, inserted.

FIG. 3 this is a section view of the cuvette holder 70, where 21, is the space for the cuvette, 22, are the fiberoptic receptacles.

FIG. 4 view of the flow thru cuvette 25, with spectrometer windows 27, being shown.

FIG. 5 this is a section view of the cuvette 25, with internal filter 26, and spectrometer windows 27, being shown.

FIG. 6 this is a section view of the smoke pickup and indicator 100, where the smoke inlet is 102 and the indicator panel is 101.

FIG. 7 this is a section view of the indicator panel 101, where there are 3 indicator LED's 103, red, 104, amber and 105, green.

FIG. 8 this is a view of an advanced design cuvette holder 70, into which disposable flow-thru cuvettes 25 are inserted.

FIG. 9 this is a section view[CC] of an advanced cuvette holder 70, where the fiberoptic cables 65 and 75 are connected via fiber connectors 76, item 77 represents the input and output optical chambers the interiors which are aluminized for reflectance, 78 is the optical path enters at 75 and exits at 65 while being directed thru the sampling chamber 27 by means of two prisms or mirrors 79, and two infrared transparent windows 85. The sample is drawn in by the input port 28 and expelled via a vacuum pump attached to exhaust port 29.

FIG. 10 this is a spectrum chart depicting the absorption of infrared energy for 3 basal cell and 1 normal tissue sample. The spectral range is 1,300 to 2,600 nm.

### DETAILED DESCRIPTION

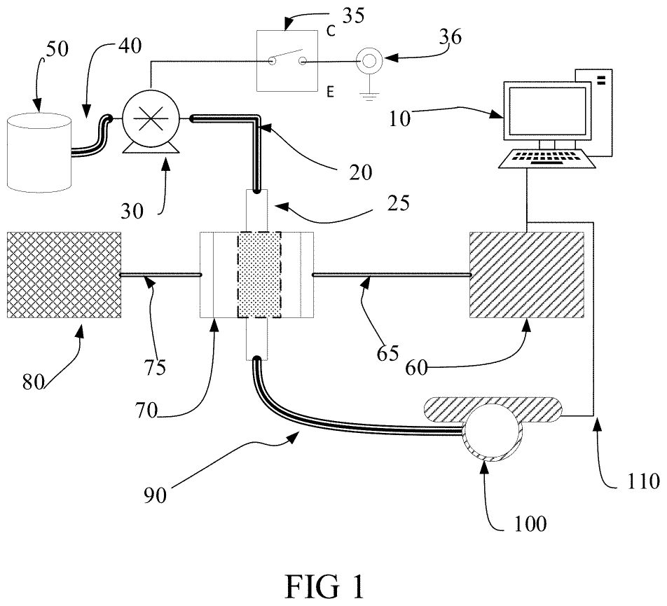

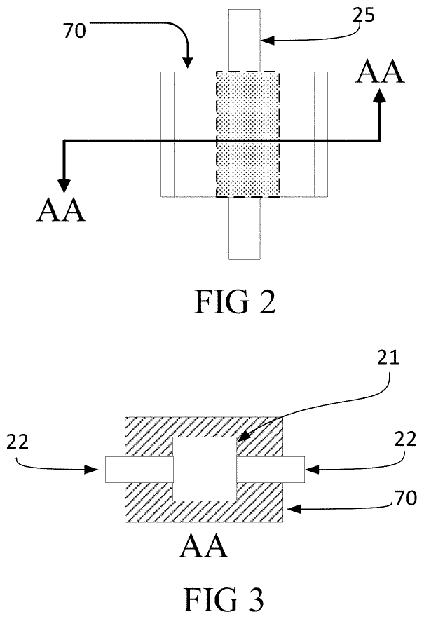

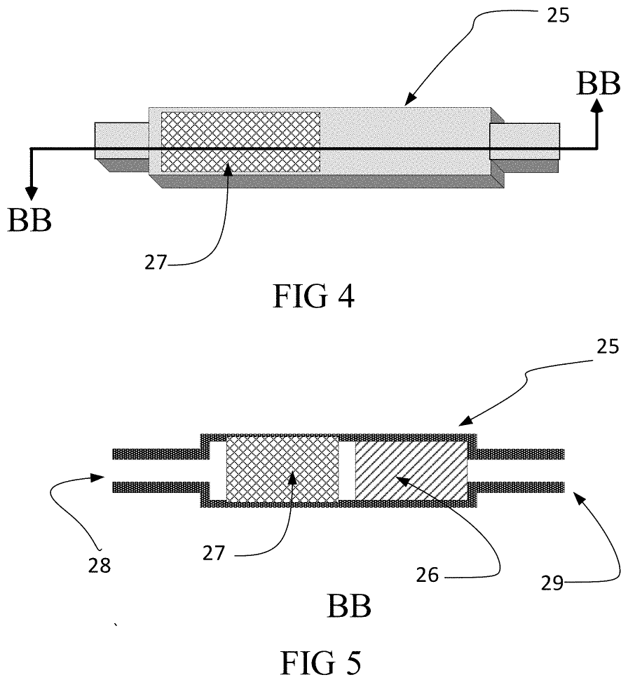

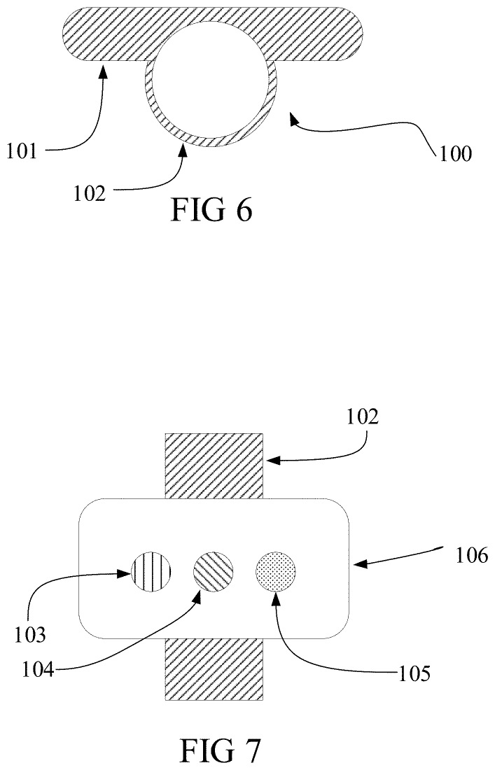

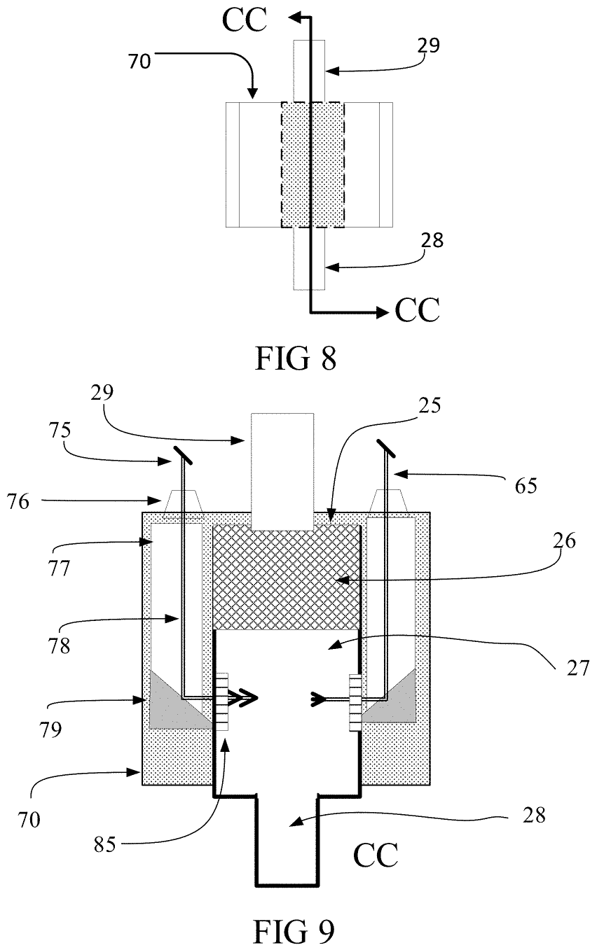

In the present invention [FIG. 1], the infrared light source 80 and the FTIR spectrometer 60 are connected by fiberoptic cables 75 and 65 respectively, to a cuvette holder 70. A windowed disposable cuvette 25 [FIG. 4] is placed in the holder completing the optical path. The system is nulled/calibrated by taking spectral samples of the empty cuvette and logging them to the computer 100. After calibration the system is ready to sample the surgical smoke.

The initial ablation is done on normal skin, the ablated smoke is evaluated by the FTIR spectrometer and the database processing software to establish a baseline. Once the baseline is established [less than 1 second] the green indicator is flashed for several second and actual surgery can begin. During surgery the indicators show green for cancerous tissue amber for questionable and red for normal tissue. In samples of normal tissue there are spectral absorption peaks at 1,680-1,750 nm, 1,880, 1,920, 2,130 and 2,500 nm. With 2,500 nm being the most pronounced. Basal cell carcinoma dose not have the previously discussed absorption bands but has its own peeks at ≈1,600-1,610 and 1,700 nm.

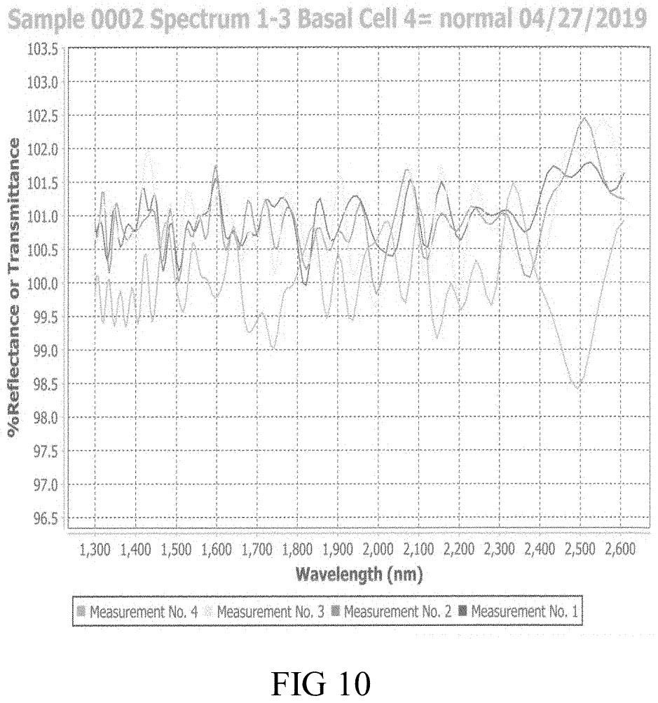

FIG. 10 shows the spectrums recorded during testing of basal cell carcinoma vs normal tissues with 3 basal cell samples and a normal tissue sample.

What is claimed is:

1. An apparatus comprising:
a) A means for collecting and sampling surgical smoke generated by tissue ablation,
b) Generating a FTIR spectral data sets in real time of the sample,
c) Logging those spectral data sets,
d) Analyzing those spectral data sets in software to determine if the smoke was generated from normal, cancerous, necrotic, or diseased tissue,
e) Provide an indicator to the surgeon in real-time [i.e. <0.5 seconds].

2. The apparatus of claim 1, wherein the means of analysis is by Artificial Intelligence [AI] software.

3. The apparatus of claim 1, wherein the means of analysis is by Inference Engines.

4. The apparatus of claim 1, wherein the means of analysis is by Neural Networks.

5. The apparatus of claim 1, wherein the means of analysis is by fuzzy C-means Clustering.

6. The apparatus of claim 1, wherein the means of software analysis is augmented by digital signal processing hardware.

7. The apparatus of claim 1, wherein the apparatus communicates with a central or remote database.

8. The apparatus of claim 1, wherein the indicator is acoustic, tactile and or visual or a combination thereof.

9. The apparatus of claim 1, wherein the indicator communicates with computers wirelessly.

10. The apparatus of claim 1, wherein the spectrometer communicates with computers wirelessly.

11. A disposable flow-thru cuvette with a particle filtering structure built in.

12. The cuvette of claim 8, wherein an electronic id component is built in.

13. The cuvette of claim 8, wherein the body is made from plastic with two parallel glass windows allow for the transmission of spectrometer light to traverse.

14. The cuvette of claim 10, wherein the windows are made of infrared transparent glass.

15. The cuvette of claim 10, wherein the windows may be made of quartz, calcium fluoride, fused silica, germanium, magnesium fluoride, potassium bromide, sapphire, silicon, sodium chloride, zinc selenide, zinc sulfide, or sapphire.
